(19) United States
(12) Patent Application Publication
(10) Pub. No.: US 2009/0223880 A2
(43) Pub. Date: Sep. 10, 2009

(54) DIALYSER

(75) Inventors: Wei Zhang, Niederwerm, DE (DE); Elke Schulte, Schweinfurt, DE (DE)

Correspondence Address: COLLARD & ROE, P.C., 1077 NORTHERN BOULEVARD, ROSLYN, NY, 11576, UNITED STATES, 516-365-9802, 516-365-9805

(73) Assignee: Fresenius Medical Care Deutschland GmbH, Bad Homburg, DE (DE)

(21) Appl. No.: 11/922,433

(22) Filed: Dec. 18, 2007

(30) Foreign Application Priority Data
Jun. 24, 2005 (DE) 10 2005 029 709.9

(51) Int. Cl. A61M 1/36; A61M 1/16; B01D 61/30

(52) U.S. Cl. 210/87; 210/85; 600/502

(57) ABSTRACT

The invention relates to a dialyser with a measuring unit assigned thereto that transmits data to the dialyser via a wireless link, the measuring unit being able to determine and transmit to the dialyser at least one measurable variable which can also be determined by another measuring device that is connected physically to the dialyser, a comparison of the at least one measurable variable as determined by the measuring unit and by the measuring device indicating whether the measuring unit is indeed the one assigned to the dialyser in question and/or whether the measuring unit is working properly.

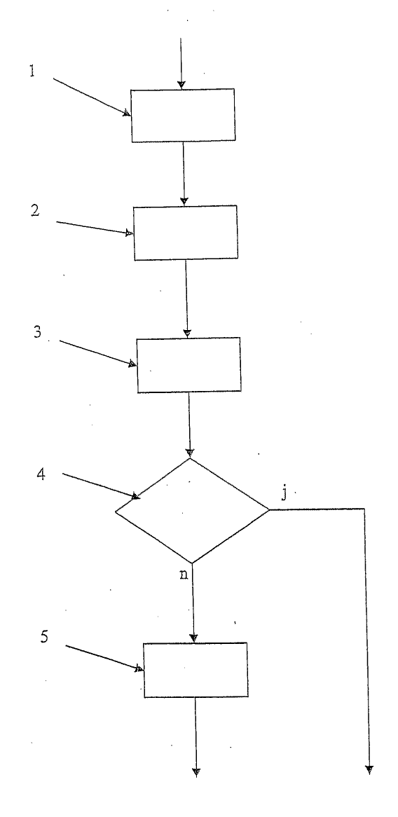

### CROSS REFERENCE TO RELATED APPLICATIONS

Applicants claim priority under 35 U.S.C. § 119 of German Application No. 10 2005 029 709.9 filed Jun. 24, 2005. Applicants also claim priority under 35 U.S.C. §365 of PCT/DE2006/001000 filed Jun. 9, 2006. The international application under PCT article 21(2) was not published in English.

The invention relates to a dialyser according to the preamble of claim 1.

Dialysers of this kind, in which the blood pressure is measured automatically by the dialyser during the dialysis treatment, are already known. A hypotonic episode during a dialysis treatment may develop suddenly. quickly and at any time, and may result in the patient's losing consciousness and/or the necessity of discontinuing the treatment session. In consequence, the patient's wellbeing and health are negatively influenced.

During a dialysis treatment, the plasma volume decreases as a result of the ultrafiltration. If the subject does not manage to restore the plasma volume from the interstitial space, the heart's filling pressure and the blood pressure sink. The higher the filtration rates are, the higher is this risk.

There are various known ways of configuring dialysers so that a drop in blood pressure can be recognized early on and/or prevented. For one, there are monitoring devices that measure the blood volume during the dialysis treatment and, if necessary, regulate the ultrafiltration. For another, there are monitoring devices that continuously monitor changes in a patient's blood pressure by determining the pulse wave transit time (PTT) and, if necessary, regulate the ultrafiltration. The time that a pressure pulse takes to travel along a patient's vessel from one point to another is a function of the blood pressure. This time can be determined with comparative ease. As start signal, an ECG signal may be used, while as stop signal, use may be made of an optical pulsometer located at a point away from the heart. This procedure has already been described in U.S. Pat. No. 6,736,789.

The method of monitoring blood pressure by means of a cuff measurement is also known. The patient may find it unpleasant that the cuff has to be “pumped up” at regular intervals (e.g. every 30 minutes) in order to perform the measurement. Another, more important, disadvantage of cuff measurements is that a radical drop in blood pressure is difficult to detect due to the measurements being discontinuous.

The PTT measuring units are normally connected to the dialyser with which the patient is to be treated. A cable connection of this kind, however, restricts the patient's ability to move, and may lead to signal falsifications when the patient does move. A further known approach in the case of measuring probes is to replace the cable connection by a radio link. This may be based, for example, on the Bluetooth Standard.

The problem addressed by this invention is to provide a means by which the dialyser can automatically verify that the measuring unit transmitting measuring data to it via a wireless link is actually the measuring unit assigned to the dialyser in question.

Especially at medical establishments in which a plurality of such dialysers are operated simultaneously, monitoring of this kind is beneficial so that each dialyser can detect whether the measuring unit transmitting measuring data to it via a wireless link is not working properly or is perhaps misconnected.

This problem is solved according to this invention by the features of claim 1, according to which, in the case of a dialyser with a measuring unit assigned thereto that transmits data to said dialyser via a wireless link, the measuring unit is able to determine at least one measurable variable and transmit the value to the dialyser, said measurable variable also being determinable by another measuring device connected physically to the dialyser, a comparison of the at least one measurable variable as determined by the measuring unit and by the measuring device indicating whether the measuring unit is indeed the one assigned to the dialyser in question and/or whether the measuring unit is working properly.

The advantage, therefore, is that the dialyser is able to monitor and check the measuring unit by independently determining a measurable variable that has also been determined by the measuring unit linked wirelessly to the dialyser. By means of a comparison, it is possible to check whether the correct measuring unit is coupled with the dialyser and/or whether the measuring unit is perhaps not working properly.

The measuring device may be physically connected to the dialyser by integrating it in the dialyser itself, or by connecting it to the dialyser by means of a cable link.

In the embodiment according to claim 2, the at least one measurable variable is the patient's pulse rate.

It is to advantage here that this measurable variable may easily be determined again independently by the dialyser. This measurable variable may also be determined with comparative ease by the measuring unit coupled wirelessly with the dialyser.

In the embodiment of the dialyser according to claim 3, the measurement by the measuring device may be performed in such manner that the pulse rate is derived from the arterial and/or venous pressure signal in the extracorporeal blood circuit.

The measured signal for the arterial and/or venous pressure is available anyway in the dialyser. It is of advantage to make further use of this measured signal in order to determine the pulse rate from it. To this end, reference is made to WO 97/10013. Preferably, the arterial pressure signal is used with preference, as here the pulse is more pronounced.

The pulse rate may be determined to advantage over a certain time interval from the pressure signal, and compared with the mean pulse-rate frequency measured over the same given time interval by the measuring unit and transmitted wirelessly to the dialyser. If these mean values are in agreement, it may be concluded that the correct measuring unit is coupled with the dialyser and that the measuring unit is working properly.

In the embodiment of the dialyser according to claim 4, the measurement by the measuring device may be performed in such manner that the pulse rate is determined by way of a cuff measurement.

The result of the measuring device connected physically to the dialyser is available in the dialyser, for example by way of a conventional blood-pressure monitor.

If at a point in time t, a cuff measurement is carried out, the pulse-rate value resulting from the measurement by the measuring device connected physically to the dialyser is compared with the value measured by the wireless measuring unit connected up to the patient. The value obtained by the measuring unit is determined at the same point in time t as for the cuff-measurement period. The value obtained by the measuring unit is the mean of the values measured by the measuring unit over this period.

If the values obtained by the measuring unit and the measuring device are in agreement, it may be concluded that the correct measuring unit is coupled with the dialyser and that the measuring unit is working properly.

In the embodiment of the dialyser according to claim 5, the at least one measurable variable is the pulse timing.

In the embodiment according to claim 5S the pulse timing determined by the measuring device may be derived, for example, from the extracorporeal pressure signal. The pulse timing is additionally determined by the measuring unit. The pulse timing as determined by the measuring device within a given time window is then compared with the pulse timing determined by the measuring unit to see if the values are in agreement, i.e. whether each pulse of the measuring device is followed or preceded in constant manner by a pulse of the measuring unit. If this is the case, it may be concluded that the correct measuring unit is coupled with the dialyser and that the measuring unit is working properly.

Evaluating the pulse timing rather than just the pulse rate has the advantage that erroneous measurements may be avoided that could arise if two patients with identical pulse rates (within metrological tolerance thresholds) are being treated, whose measuring units have been mixed up. The likelihood of two patients having not only the same pulse rate but also the same pulse timing is significantly smaller.

Normally, verification may be performed at the commencement of a dialysis session. It is equally possible to repeat this verification procedure periodically—every 30 minutes, for example. However, verification may also be performed with comparative ease during dialysis, since the appropriate measurable variables are easily available.

The principle of how a dialyser according to the invention works is shown in the form of an example in the drawing.

Block 1 represents the commencement of a dialysis session. The data values for the measuring device and the measuring unit are initialised, i.e. set to “0”.

Subsequently, in step 2, the \'ariable is determined by the measuring device, which is physically connected to the dialyser.

In step 3, the same variable is determined by the measuring unit, which transmits the data wirelessly to the dialyser.

In step 4, the difference between the values obtained for the measurable variable by the measuring device and the measuring unit is checked to see whether it is smaller than a predetermined threshold value.

If this is the case, the measuring unit coupled with the dialyser is acknowledged as being the correct one.

If not, the possibility that the measuring unit is connected to the wrong patient is acknowledged. A warning according to step 5 then ensues.

Step 5 can be varied to the effect that an identifier is provided for counting the instances in which, in step 4, a deviation above the threshold value is detected. Only if this identifier reaches a characteristic threshold is the fault signal generated. This measure makes it possible to prevent individual erroneous measurements from causing inappropriate warning signals. if, by contrast, the subsequent values determined by the measuring device and the measuring unit are in agreement, the identifier may be reset—either immediately or on fulfillment of certain criteria (e.g. a minimum number of measurements that are in agreement).

1. A dialyser with a measuring unit assigned thereto, the data transmission from the measuring unit to the dialyser ensuing wirelessly, wherein the measuring unit is able to determine and transmit to the dialyser at least one measurable variable (3) which can also be determined by another measuring device that is physically connected to the dialyser (2), a comparison of the at least one measurable variable as determined by the measuring unit and by the measuring device indicating whether the measuring unit is indeed the one assigned to the dialyser in question and/or whether the measuring unit is working properly (4).

2. The dialyser according to claim 1, wherein the at least one measurable variable is the patient's pulse rate.

3. The dialyser according to claim 2, wherein the measurement by the measuring device can be performed in such manner that the pulse rate is derived from the arterial and/or venous pressure signal in the extracorporeal blood circuit of the dialyser.

4. The dialyser according to claim 2, wherein the measurement by the measuring device can be performed in such manner that the pulse rate is determined by way of a cuff measurement.

5. The dialyser according to claim 1, wherein the at least one measurable variable is the pulse timing.
